(19) United States
(12) Patent Application Publication
(10) Pub. No.: US 2014/0259598 A1
(43) Pub. Date: Sep. 18, 2014

(54) ANALYSIS OF LOCALIZED WASTE MATERIAL

(71) Applicant: SOLAR TURBINES INCORPORATED, San Diego, CA (US)

(72) Inventors: Mark D. Lipschutz, San Diego, CA (US); Alejandro Manriquez, San Diego, CA (US)

(73) Assignee: SOLAR TURBINES INCORPORATED, San Diego, CA (US)

(21) Appl. No.: 14/286,675

(22) Filed: May 23, 2014

Related U.S. Application Data
(63) Continuation of application No. 13/398,054, filed on Feb. 16, 2012.

(51) Int. Cl. G01N 33/20; G01M 5/00

(52) U.S. Cl. CPC G01N 33/20; G01M 5/0016; G01M 5/0033
USPC 29/407.01

(57) ABSTRACT

A testing method for a component is provided. The method identifies a flaw region of the component. The flaw region is prone to defects. The method then isolates a waste material associated with the identified flaw region. The method analyzes the isolated waste material for an undesirable microstructure associated with defects. Subsequently, the method determines rejection and acceptance of the component based, at least in part, on the analysis.

This application is a continuation of U.S. patent application Ser. No. 13/398,054, filed on Feb. 16, 2012.

### TECHNICAL FIELD

The present disclosure relates to a non-destructive manufacturing and inspection method, and more particularly to metallurgical analysis of localized waste material for testing.

### BACKGROUND

Non-destructive testing of parts prior to their application in service is essential to assess the quality of the part to facilitate in early detection of high risk parts. U.S. Pat. No. 7,757,364 relates to achieving improved ultrasonic testing coverage of a finished machined component by modifying a finished machine component forging for ultrasonic inspection. The invention involves constructing a forging envelope surrounding a machine component forging. Materials are added to the forging envelope to facilitate inspection.

### SUMMARY OF THE DISCLOSURE

In one aspect of the present disclosure a method for testing a component is provided. The method identifies a flaw region of the component. The flaw region is prone to defects. The method then isolates a waste material associated with the identified flaw region. The method analyzes the isolated waste material for an undesirable microstructure associated with defects. Subsequently, the method determines rejection and acceptance of the component based, at least in part, on the analysis.

Other features and aspects of this disclosure will be apparent from the following description and the accompanying drawings.

### BRIEF DESCRIPTION OF THE DRAWINGS

FIG. 1 is a diagrammatic view of an exemplary forging compressor blade having a defect, according to one embodiment of the disclosure; and

FIG. 2 is a process for testing a waste material associated with the compressor blade.

### DETAILED DESCRIPTION

FIG. 1 illustrates an exemplary component 100 which may be, as shown, a compressor blade 102 during a forging manufacturing process. The compressor blade 102 may be used in a gas turbine engine, an axial flow compressor, and the like. In some situations, the compressor blade 102 may have a defect (not shown in figure). The defect may at a later stage lead to the failure of the compressor blade 102. The defect may be too small to be detected by known non-destructive testing methods.

Moreover, a waste material 106 may be associated with the compressor blade 102, as shown in FIG. 1. In one embodiment, the waste material 106 may include forging flash. Close inspection of FIG. 1 shows that the geometry of the compressor blade 102 is such that there is a transition in thickness of the compressor blade 102 from a root 108 to an airfoil 110 of the compressor blade 102. The defect may be located at this transitioning area, which may be at the leading edge root end of the airfoil 110 as indicated in FIG. 1. Regions such as these of the compressor blade 102 may represent a flaw region 112, due to an increased tendency of having an internal flaw. The flaw region 112 illustrated in FIG. 1 is on an exemplary basis. The compressor blade 102 may include other such areas prone to having defects.

The presence of a fine grain microstructure 114 or other undesirable microstructure in the waste material 106 may be indicative of a lower fatigue durability of the compressor blade 102. FIG. 1 shows a test material 116 adjacent to the flaw region 112. The test material 116 can then be examined for the presence of the undesirable microstructure using destructive testing techniques while maintaining the integrity of the component 100.

The disclosure relates to an inspection or testing method 200 in which the test material 116 is analyzed in order to determine if the corresponding component 100 will have a tendency to be subjected to fatigue failure in the future. The method 200 will be described in detail in connection with FIG. 2.

### INDUSTRIAL APPLICABILITY

The fatigue failure tendency of the component 100 may be linked to the presence of the fine grain microstructure 114 or the other undesirable microstructures on the compressor blade 102. However, the fine grain microstructures 114 and/or the undesirable microstructures are sometimes too small to be detected by standard non-destructive evaluation techniques, thereby causing inability of standard non-destructive evaluation techniques to identify at-risk components. Fatigue cracks may initiate at the pre-existing defects in the compressor blade 102 near the leading edge root end of the airfoil 110 and may have a tendency to propagate until overload separation of the airfoil 110 occurred. However, no technique existed to detect the presence of these minute defects.

As shown in FIG. 2, the disclosure provides a method 200 for determining a tendency of failure of the compressor blade 102, based on the presence of known microstructures in the waste material 106 associated with the flaw region 112 of the compressor blade 102 or other component 100.

Initially at step 202, the flaw region 112 of the compressor blade 102 is identified. The flaw region 112 may include that region of the compressor blade 102 which is prone to exhibit the defect. In one embodiment, the flaw region 112 may include the leading edge root end of the airfoil 110 of the compressor blade 102. In another embodiment, the flaw region 112 may include the region of the compressor blade 102 which has a variation or transition in thickness of the material used to form the compressor blade 102. A person of ordinary skill in the art will appreciate that the flaw region 112 may additionally include other areas of the component 100 which are prone to have the defect.

At step 204, the waste material 106 associated with the identified flaw region 112 is isolated. In one embodiment, the waste material 106 may include forging flash. The waste material 106 may be adjacent to the flaw region 112 identified in step 202. After the final forging compressor blade 102 is produced, the forging flash may be removed by any suitable method. Subsequently, at step 206, the isolated waste material 106 is analyzed for the presence of the known microstructure.

In one embodiment, analysis of the isolated waste material 106 may involve detecting the presence of microstructures which are known to be associated with defects that can cause failure of the component 100, in the isolated waste material 106. The analysis may involve metallographically evaluating the waste material 106 to characterize the microstructure and detect the presence of the known microstructure. A person of ordinary skill in the art will appreciate that the known microstructures may include the fine grain microstructures 114 that are found in high risk components.

Following the above, in step 208, rejection and acceptance of the component 100 is determined based on the analysis conducted in step 206. If the known microstructure is detected in the isolated waste material 106, then the compressor blade 102 is said to exhibit high risk of failure. Accordingly, the said compressor blade 102 may be rejected prior to usage. In another embodiment, a tendency of failure of the component 100 may be determined based on the presence of the known microstructure in the component 100.

In yet another embodiment, the method 200 may be used to inspect the component 100 or the compressor blade 102 once the test material 116 was identified. In this case, the method 200 analyzes the waste material 106 of the component 100 for presence of the undesirable microstructure or the fine grain microstructure 114. Subsequently, the method determines the rejection and acceptance of the component 100, as in step 208.

In the method 200 only the waste material 106 associated with the component 100 is tested, while the remainder of the component 100 remains intact. Moreover, the description provided above in relation to the testing of the compressor blade 102 is merely on an exemplary basis and does not limit the scope of the disclosure. A person of ordinary skill in the art will appreciate that the method 200 may be employed in any number of industries which utilize forgings, without any limitation.

While aspects of the present disclosure have been particularly shown and described with reference to the embodiments above, it will be understood by those skilled in the art that various additional embodiments may be contemplated by the modification of the disclosed machines, systems and methods without departing from the spirit and scope of what is disclosed. Such embodiments should be understood to fall within the scope of the present disclosure as determined based upon the claims and any equivalents thereof.

What is claimed is:

1. A method of manufacturing comprising:
forging a component;
identifying a flaw region of the component, wherein the flaw region is prone to defects;
removing a waste material associated with the identified flaw region, the waste material created during the forming of the component;
isolating a test material from the waste material associated with the identified flaw region;
analyzing the isolated test material for an undesirable microstructure associated with defects; and
determining rejection and acceptance of the component based, at least in part, on the analysis.

2. The method of claim 1, wherein the component is a compressor blade.

3. The method of claim 1, wherein the flaw region is an area of the component having a transition in thickness.

4. The method of claim 1, wherein the flaw region includes a leading edge root end of an airfoil of the compressor blade.

5. The method of claim 1, wherein the waste material is flashing from a forging process.

6. The method of claim 1, wherein the waste material associated with the identified region is adjacent to the identified region.

7. The method of claim 1 further including determining a tendency of failure of the component.

8. The method of claim 1, wherein the undesirable microstructure is a fine grain microstructure.

9. A method of inspecting a component during a manufacturing process, the method comprising:
forming a component;
isolating a test material from a waste material adjacent to an identified flaw region of the component, the waste material created during the forming of the component;
analyzing the test material for presence of an undesirable microstructure; and
determining rejection and acceptance of the component based, at least in part, on the analysis.

10. The method of claim 9, wherein the component is a compressor blade.

11. The method of claim 9 further including analyzing a waste material at an area of the component having a transition in thickness.

12. The method of claim 9 further including analyzing a waste material at a leading edge root end of an airfoil of the compressor blade.

13. The method of claim 9, wherein the waste material is flashing from a forging process.

14. The method of claim 9 further including determining a tendency of failure of the component based, at least in part, on the rejection.

15. The method of claim 9, wherein the undesirable microstructure is a fine grain microstructure.

16. A method of manufacturing comprising:
forging a component;
identifying a flaw region of the component, wherein the flaw region is prone to defects;
removing a forging flash associated with the identified flaw region, the forging flash created during the forming of the component;
isolating a test material from the forging flash;
metallographically analyzing the isolated test material for the presence of an undesirable microstructure; and
determining rejection and acceptance of the component based, at least in part, on the analysis.

17. The method of claim 16, wherein the undesirable microstructure is a fine grain microstructure.

18. The method of claim 16, wherein the isolating of the test material and the removing of the forging flash are non-destructive to the component.

19. The method of claim 16, further including determining a tendency of failure of the component based on the presence of the undesirable microstructure.

20. The method of claim 16, wherein the component includes an airfoil, and wherein the forging flash is at a leading edge root end of the airfoil.
